(19) United States
(12) Patent Application Publication
(10) Pub. No.: US 2022/0296809 A1
(43) Pub. Date: Sep. 22, 2022

(54) PARENTERAL ADMINISTRATION OF MEDICATIONS, FLUIDS, AND NUTRITION

(71) Applicant: Ilana Katsnelson, El Sobrante, CA (US)

(72) Inventor: Ilana Katsnelson, El Sobrante, CA (US)

(21) Appl. No.: 17/698,337

(22) Filed: Mar. 18, 2022

Related U.S. Application Data
(60) Provisional application No. 63/161,709, filed on Mar. 16, 2021.

(51) Int. Cl. A61M 5/142; A61M 25/10; A61K 31/485; A61K 31/325; A61M 5/148; A61L 31/04; A61L 31/10

(52) U.S. Cl. CPC A61M 5/14276; A61M 25/10; A61K 31/485; A61K 31/325; A61M 5/148; A61L 31/047; A61L 31/10; A61M 2005/1405

(57) ABSTRACT

The present invention relates to the delivery of therapeutic agents into interstitial fluid and designed to substitute other forms of parenteral administration of fluid and medications when it is appropriate. The invention features an expandable reservoir made from mesh and a stylet guided catheter. The innovation of proposed catheter comprises to a catheter for administration of fluid and medications and a mechanism that safely secures the catheter inside the reservoir in order to prevent it from accidental dislodging. One of the ways to secure catheter is self-inflated balloon on its proximal end of the catheter.

### CROSS-REFERENCE TO RELATED APPLICATIONS

The present application claims priority to U.S. Provisional Patent Application No. 63/161,709 filed Mar. 16, 202, entitled “NEW WAY TO PARENTERAL ADMINISTER MEDICATIONS, FLUIDS AND NUTRITION” which is hereby incorporated by reference in its entirety.

### TECHNICAL FIELD

The present invention is generally related to new ways of parenteral administration of fluids and medications as well as the administration of medications via medical implant (pump) devices.

### BACKGROUND

There are currently multiple ways for the parenteral administration of medications and fluids and the most common are: subcutaneous (S/C), intravenous (IV), and intramuscular (IM). IV drug and fluid administration can be further subdivided into a peripheral vein and central vein administration. Each method has its own advantages and disadvantages. New interstitial fluid administration via expandable reservoir that we describe in this patent has advantages over current methods for the parenteral administration of medications and fluids in some applications.

The IV route is used most often among parenteral routes of fluid administration. However, IV administration requires highly qualified medical personnel. It requires a lot of skill to find the vein, make sure that the vein does not become perforated, and so on. Qualified medical personnel is also required to maintain IV access for the duration of an infusion. Prolonged use of the vein for administration of medications usually results in vein sclerosis. As a result, patients who require prolonged IV administration of fluid or medications often require central vein placement or a central venous catheter (CVC) inserted into a large vein. CVC is often done by a radiologist under the guidance of ultrasound. The immediate risks of peripherally inserted CVC include: injury to local structures, phlebitis at the insertion site, air embolism, hematoma, arrhythmia, and catheter malposition. Late complications can also include infection, thrombosis, and catheter malposition.

Because of the risks involved, additional parenteral routs for the administration of fluid and medications would be beneficial and preferred for many patients. Use of the expandable reservoir for the administration of fluid and medications will require much less time to install and only minimal maintenance after installation. The interstitial fluid-filled reservoir would be also very beneficial in the case of a mass casualty incident (MCI) when a large number of patients require treatment simultaneously and there is not enough medical personnel to provide required medical care.

In the case of an MCI, a significant number of patients die from hypovolemic shock, and survival rates will significantly improve with timely administration of fluid and pain medications. Similar scenarios occur in the developed world when there is an epidemic of diarrheal disease and children are dying from dehydration. Because it will take much less time to install and maintain the expandable reservoir the issue of a mismatch between the number of patients that require urgent medical care and the number of medical personnel available to provide it will be resolved. The expandable reservoir will be able to substitute the central vein or umbilical vein catheter for the administration of parenteral nutrition and medications for prematurely born babies.

Parenteral drug administration via expandable reservoir can replace IV administration in many cases. For example, if a patient requires long-term IV antibiotic treatment for urosepsis or long-term IV administration of other medications. In some cases, the use of an expandable reservoir would allow patients to receive medical care at home rather than in the hospital or skilled nursing facility. This technology would decrease the cost of medical care and can save lives.

Another important use for expandable or non-explainable reservoirs could be its use with implantable, reloadable drug delivery systems and pumps. These medical devices would help to address treatment compliance in patients with mental and cognitive deficits. The most important indication for the implantable and reloadable medication pumps will be the treatment of substance use disorder (SUD) or drug abuse. In the case of SUDs, it is the disease itself that prevents patients from being compliant with treatment and no medication is effective if a patient is failing to take it.

The reloadable and implantable medicine pumps are already used in medical practice. Rechargeable devices store and deliver medications into an intrathecal space (intrathecal delivery system) for treatment of chronic pain and spasticity. Examples of this technology are Baclofen Pump or opioid pain pumps that deliver medications for pain management into intrathecal space. One of the examples is Synchromed™ Intrathecal Pump. In these cases, medications are delivered directly into the cerebrospinal fluid (CSF). As a result, the formation of scar tissue around the catheter is avoided. Unfortunately, reloadable implantable medical pumps that deliver medications outside CSF are not widely used in medical practice. The formation of a “capsule” of scar tissue around any kind of implant “foreign body” is a normal part of the healing process. The body automatically reacts to any foreign object it detects and attempts to isolate the object by creating a barrier of scar tissue around it. Scar tissue prevents the administration of a precise dose, which is the main requirement for these medical devices. Attaching drug-eluting expandable or non-expandable reservoirs to the rechargeable implant delivery system would address the issue of scar formation and precise dose administration. Drug-eluting reservoirs slowly emit medication that prevents scar formation. The implantable medication pump will administer medication into the open space that the reservoir created. Even with the formation of some scar tissue around the device, it would be difficult to seal the large surface of the reservoir compared to small openings on the implantable device itself.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

The embodiments provided herein relate to the delivery of therapeutic agents into interstitial fluid and are designed to substitute other forms of parenteral administration of fluid and medications when it is appropriate. The invention features an expandable reservoir made from mesh (1) and a stylet guided catheter (2). The innovation of the proposed catheter comprises a) catheter for administration of fluid and medications and 2) a mechanism that safely secures the catheter inside the reservoir to prevent it from accidental dislodging. One of the ways to secure a catheter is a self-inflated balloon on its proximal end of the catheter.

The advantage of the proposed system is that the catheter can be connected not only to a proprietary infusion system but also to any currently available infusion systems. One of the examples is the patient-controlled anesthesia (PCA) pumps. Depending on different indications, duration of treatment and other factors mesh for the expandable reservoir can be made from different materials. For example, it can be made from synthetic or natural materials, as well as metals. Indication and duration of treatment will be determining factors if mesh should be made from biodegradable material. Depending on the indication, the mesh could also be eluded with medications. The medication eluded mesh can be used to increase absorption, prevent the proliferation of fibrosis tissue around the reservoir, or prevent infection.

Another part of this invention is attaching a drug eluded mesh reservoir to the implantable drug delivery pump to address scar and fibrous tissue formation around the pump. The scar tissue prevents precise dose administration which is critical for the efficacy of treatment of the diseases.

Drug delivery is a very important area of delivering medical care to patients. As a result, multiple prior arts are related to this field. The majority of the prior art medical devices are designed to work with proprietary infusion systems that are also a part of each particular patent. The advantage of the proposed invention is its ability to work with commercially available drug infusion systems and PCAs. Another major distinction is expandable reservoirs made from mesh with a port on the proximal end of the reservoir. The catheter that is inserted and removed from the reservoir as needed decreases the risk of infection due to constant exposure to the external world.

### BRIEF DESCRIPTION OF THE DRAWINGS

A more complete understanding of the embodiments, and the attendant advantages and features thereof, will be more readily understood by reference s—to the following detailed descriptions when considered in conjunction with the accompanying drawings wherein:

FIG. 1A illustrates a collapsed reservoir with the balloon inflating device, according to some embodiments;

FIG. 1B illustrates an expanded reservoir with the balloon inflating device, according to some embodiments;

FIG. 2A illustrates a mesh in collapsed form and expanded form, according to some embodiments;

FIG. 2B illustrates an expanded reservoir, according to some embodiments;

FIG. 3A illustrates a stylet with a catheter and a hub with two ports in addition to an inflatable balloon port and a port for administration of fluid or medications, according to some embodiments;

FIG. 3B illustrates a catheter with a hub for the administration of fluid or medications with an inflated balloon, according to some embodiments;

FIG. 3C illustrates a catheter with an inflated balloon inside an expanded reservoir, according to some embodiments;

FIG. 4A illustrates an expanded reservoir with a catheter attached to an infusion system, according to some embodiments;

FIG. 4B illustrates an expanded reservoir with a catheter attached to an infusion system attached to the human body, according to some embodiments;

FIG. 5 illustrates an expanded catheter attached to patient-controlled anesthesia (PCA) pump, according to some embodiments;

FIG. 6A illustrates a medication pump, according to some embodiments;

FIG. 6B illustrates a medication pump, according to some embodiments;

FIG. 6C illustrates a medication pump with a mesh reservoir attached, according to some embodiments; and

FIG. 7 illustrates a medication pump under abdominal skin, according to some embodiments.

### DETAILED DESCRIPTION

The specific details of the single embodiment or variety of embodiments described herein are outlined in this application. Any specific details of the embodiments are used for demonstration purposes only, and no unnecessary limitations or inferences are to be understood therefrom.

Before describing in detail exemplary embodiments, it is noted that the embodiments reside primarily in combinations of components related to the system. Accordingly, the device components have been represented where appropriate by conventional symbols in the drawings, showing only those specific details that are pertinent to understanding the embodiments of the present disclosure, so as not to obscure the disclosure with details other than those readily apparent to those of ordinary skill in the art having the benefit of the description herein.

As used herein, the term “mesh” refers to an interlaced structure that is created by the network of wires or threads.

As used herein, the term “reservoir” refers to a receptacle or part of a medical device designed to hold interstitial fluid.

As used herein, the term “expandable reservoir” refers to a device with permeable walls that are inserted into the human body when it is in a small collapsed form and then is expanded after insertion.

As used herein, the term “balloon technology” refers to an inflatable balloon inside a patient body that expands when air is pumped into the balloon.

As used herein, the term “biodegradable material” refers to material inserted into the human body that dissolves/disappears over time, so that there is no need for a separate procedure to remove a device that is made out of it.

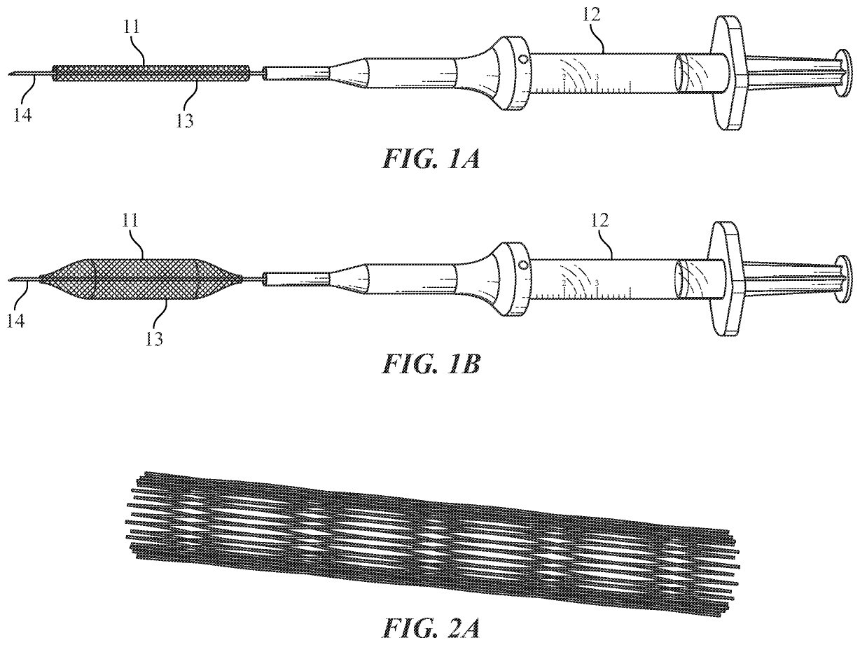

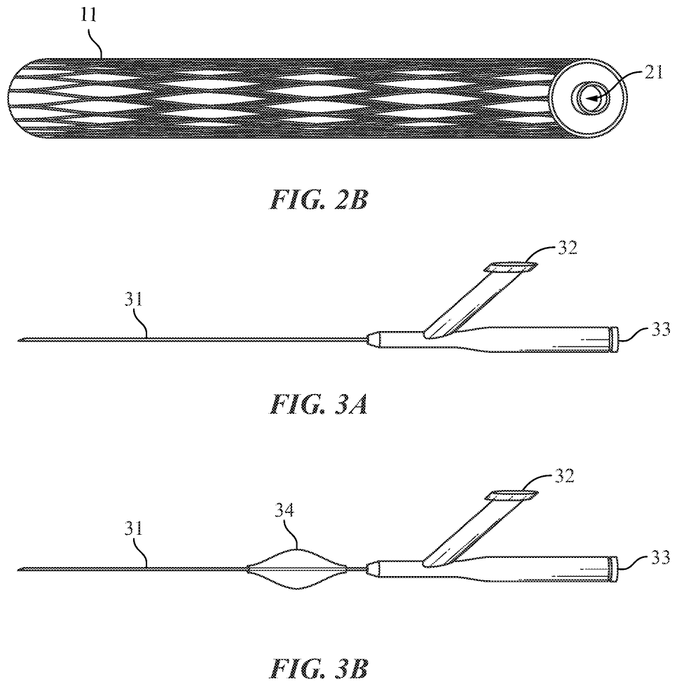

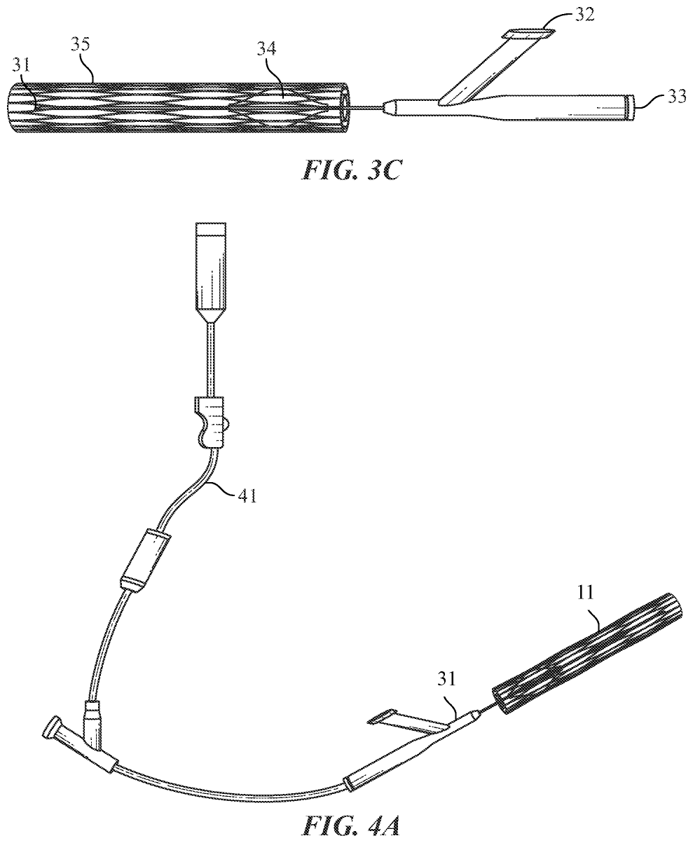

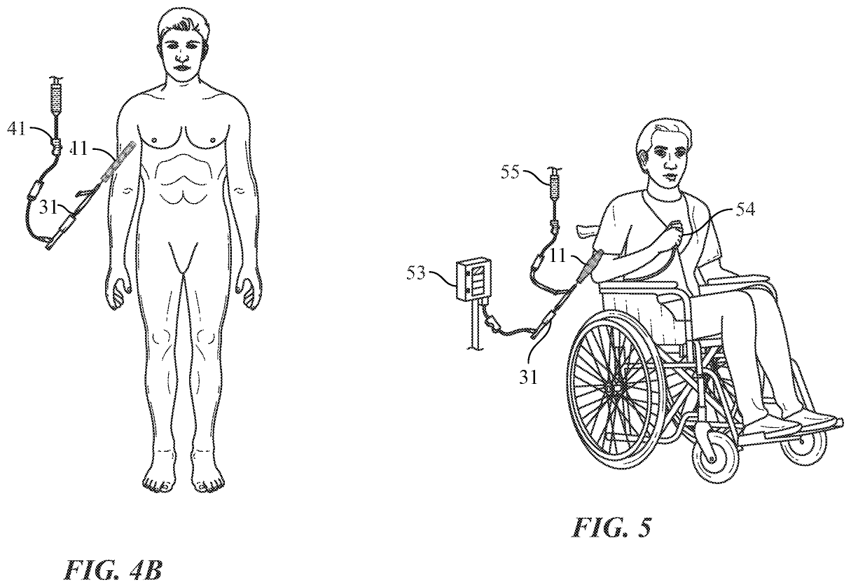

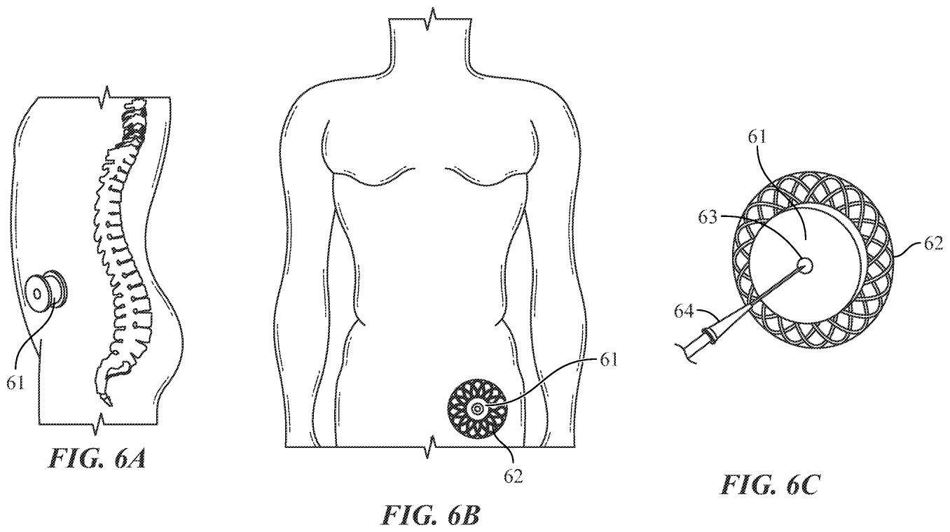

An expandable reservoir can be inserted into several parts of the body, for example, the arms legs, or abdomen. The expandable reservoir is inserted in a collapsed form under local anesthesia in a different area of the body (as shown in FIG. 1A). After insertion, the reservoir is expanded using balloon technology or other methods (as shown in FIG. 1B). The balloon will be deflated and removed after the expansion of the reservoir is completed. Expansion of the reservoir creates an open space inside the reservoir. Once the reservoir is expanded, the stylet guided catheter (as shown in FIG. 3A-B) is inserted into the reservoir through the reservoir's port opening on the proximal end (as shown in FIGS. 2A and 2B). The catheter is then secured inside the reservoir to ensure that a patient cannot accidentally remove it. FIG. 4A-B illustrates several techniques which could be used to secure the catheter in place. For example, an inflatable balloon can be used to secure the catheter inside the reservoir. The balloon which in its size is bigger than the reservoir port would prevent accidental removal of the catheter. In addition to the mechanism that secures the catheter inside the reservoir, the catheter also has a medication port for the administration of fluids and medications. The medication port of the catheter is connected to an infusion system as shown in FIG. 5 and FIG. 6. After administration of fluids, or medications through the catheter, the small balloon can be deflated, and the catheter safely removed. Removal of the catheter when it is not used will significantly decrease the risk of infection because there is no exposure to the outside world. The single-use catheter can be safely reinstated if further drug administration is needed.

FIG. 1A illustrates a collapsed reservoir with the balloon inflating device. The collapsed reservoir 11 includes a stylet 14. The collapsed reservoir 11 may be constructed of mesh 13. A syringe 12 or other device is connected to the collapsed reservoir 11 to provide inflation as shown in FIG. 1B which illustrates an expanded reservoir 11.

FIGS. 2A and 2B illustrate the mesh in collapsed form and expanded form. Port 21 is positioned in the reservoir and allows a catheter to be inserted therein for the administration of fluid and medications.

FIG. 3A illustrates a stylet with a catheter and a hub with two ports in addition to an inflatable balloon port and a port for administration of fluid or medications. A needle 31 is in communication with a balloon inflation port 32 as well as a hub 33 for the administration of medication and/or fluids. FIG. 3B illustrates a catheter (needle) 31 with a hub 33 for the administration of fluid or medications with an inflated balloon 34. FIG. 3C illustrates a catheter (needle) 31 with inflated balloon 34 inside an expanded reservoir 35.

FIG. 4A illustrates an expanded reservoir 11 with a catheter 31 attached to an infusion system 41. FIG. 4B illustrates an expanded reservoir 11 with a catheter 31 attached to an infusion system 41 attached to the human body.

FIG. 5 illustrates an expanded reservoir 11 catheter 31 attached to patient-controlled anesthesia (PCA) pump 53 having a control 54 including an IV fluid administration system 55 in communication thereto.

FIG. 6A-6C illustrates an implantable medication pump 61 and mesh 62 attached thereto. FIG. 6B illustrates the implantable medication pump 61 having the mesh 62 and second port 63 for the refill of medication via a medication refill infusion system 64.

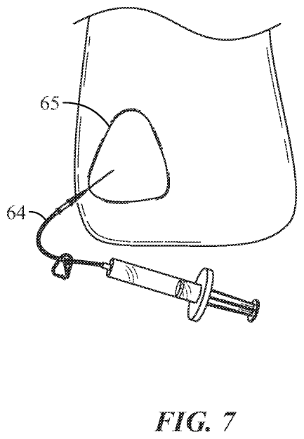

FIG. 7 illustrates a medication pump under abdominal skin having a medication refill infusion system 64 and a medication pump 65 positioned under the abdominal skin.

### Material of the Reservoir

The selection of material used for the reservoir should be contingent on the indication and duration of treatment. Another important consideration would be the need to remove the expandable reservoir at the end of treatment. For example, if the reservoir is used in a hospice setting with PCA the removal of the reservoir is not necessary. Reservoirs can be made from other appropriate materials including biodegradable polymers, synthetic or natural materials, or metal depending on the indication. Biodegradable material or polymers are defined as capable of being broken down into innocuous products by the action of the human body. The reservoir can be eluted with medications if desired. The material selected for the reservoir should not cause a negative reaction in humans. Similarly, the drug-eluting reservoirs are designed to suppress the formation of collagen and fibrosis tissue around the reservoir. A drug-eluting reservoir will emit a drug over time.

For diseases that require a relatively short duration of treatment, biodegradable reservoirs from biodegradable material bare or eluted with medications would be preferred. One example of short use of reservoirs would be the treatment of dehydration caused by diarrhea or hypovolemic shock. Another indication would require a much longer duration for treatment for example use of antibiotics or long-term parenteral nutrition may use materials that are not degraded.

If a metal wire is the most convenient material for the reservoir due to indication, metal should be selected from metals that are already used in other medical devices, for example, in cardiovascular stents. The advantage of using metals that are already used in other class 3 medical devices is their proven safety record. These metals include (but are not limited to) L-605 Cobalt chromium, Cobalt alloy, F-562 Cobalt chromium, 316 L Stainless steel, Platinum chromium, 316 L Stainless steel (iron, chromium, nickel, molybdenum).

Preferably, the medication in the drug-eluting reservoirs could be selected from medications that already have a proven record of inhibiting the proliferation of scar tissues, such as sirolimus, everolimus, umirolimus, novolimus, and paclitaxel. These medications are successfully used in cardiac drug-eluded stents for the same reason. However other medications that inhibit scar formation can be used as well. For example, drug-eluting reservoirs can also use enzyme hyaluronidase. Hyaluronidase “decomplex” hyaluronic acid is an essential component of the extracellular matrix (ECM). Hyaluronidase enhances the diffusion capacity and bioavailability of injected drugs.

Examples of biodegradable materials that can be used in biodegradable reservoirs are polyglactin 910 or Antibacterial (polyglactin 910), Monocryl or Monocryl antibacterial, or made of organic materials for example chromic gut.

### Subcutaneous Fluid Administration Vs Administration of Fluid Via the Reservoir

Subcutaneous (SC) route of fluid and medication administration can be used in the management of unwell older people who have poor venous access, or who are unable to tolerate intravenous cannulation, which presents a common and difficult challenge for clinicians (Barton et all 2004). In some cases, subcutaneous infusions SC (hypodermoclysis) is used in palliative care when IV access is difficult. However, the volume of fluid that can be administered SC is significantly lower compared to IV fluid administration. The rate of fluid administration with hypodermoclysis is usually 50 to 60 ml/hour (Barton et all 2004, Caccialanza et all 2018). Elderly skin also has much less elasticity which makes administration of fluid much easier which is not the case with younger patients. SC fluid administration is also requiring significant nursing supervision as the needle can be easily dislodged. In many cases, the administration of fluid via an expandable reservoir would be a better option.

The absorption of fluid, Total Parenteral Nutrition (TPN), and medications after IM, SC, or expandable reservoir is based on passive and active diffusion. Active diffusion is medication specific. However, the passive diffusion rate follows Fick's law of diffusion. The rate of diffusion is directly proportional to the surface area and concentration difference.

Diffusion rate=Surface area*concentration gradient/thickness of the membrane.

A significant increase in surface for absorption dramatically increases the rate of fluid, TPN, or medication that can be delivered in the time interval.

With S/C injection, absorption surface area is about equal to the end of the needle.

However, the assumption can be made that a small bubble is formed around the needle and the surface area of the 0.5 cm bubble radius. The volume of fluid that can be administered S/C is 1 cc/min or 62 cc/h Caccialanza et all 2018, Surface area of the sphere at the end of a needle is:
A=4πr²A=A=4π1²=4π cm² and its volume V=4/3πr3V=4/3*3.14*13.

Surface area of the reservoir is significantly larger and depends on its size. Surface area of cylinder 1 cm wide over 4 cm long is:
A=2πr² A=2π1²=2π cm² Surface area without top and bottom S=4 cm*2π=8π cm² Adding top and bottom A=2π*2=4π cm² Total surface area will be 4π+8π=12π cm²
   Cylinder volume=A=2π1²*hA=2π cm²*4 cm=8π cm3

The surface area increases as the cylinder size get bigger:

TABLE 1
Table 1. Comparison of the surface area of two different
sizes of cylinders and S/C fluid administration.

| | Sphere 0.5 cm in diameter | | | | | |
|---|---|---|---|---|---|---|
| | (hypodermoclysis) | | Cylinder 1 cm * 4 cm | | Cylinder 1 cm * 5 cm | |
| Surface area | 12.56 | cm² | 37.68 | cm² | 43.96 | cm² |
| Volume | 4.18 | cm3 | 25.12 | cm3 | 31.4 | cm3 |

| | | | |
|---|---|---|---|
| Fluid administration | 1 cc/min or 60 cc/h | 3 cc/min or 180 cc/h** | 3.5 cc min or 210 cc/h** |

The absorption will be further enhanced if fluid is administered under physiologic positive pressure. The amount of fluid administered via an expandable reservoir should be sufficient to treat moderate to severe dehydration in children in the developing world. In more severe cases of dehydration, when a large volume of fluid is required over a relatively short period two reservoirs in two different sites or larger sizes of the reservoir can be used. An advantage of using the reservoir approach vs. IV fluid is that reservoirs can be placed very quickly and they will not need nursing staff to be maintained.

Dehydration decreases the fluid component of the blood is which, in turn, increases the oncotic pressure in the capillary. That would improve absorption even further, as oncotic pressure plays important role in the reabsorption of fluid from tissue back to the vascular system.

Pressure difference: Pressure diffidence is another factor that increases absorption. Increased pressure inside the reservoir also increases the movement of medications and fluid into general circulation. In many cases when sophisticated equipment is not available, the water gravity created by an iv fluid bag hung on the pole. delivers the medication into the IV line at a safe and steady rate.

Determining Water Pressure. Use the formula pressure (P)=0.43 x-height of the water column in feet (h). We use the constant 0.43 (lb/in\hat{ }2)/ft because this is the amount of pressure 1 foot of water places on a surface below it regardless of the volume of water.

However, to increases absorption, fluid can be administered with pressure similar to arterial end net filtration pressure to closely mimic arterial end net filtration pressure. The pressure used for fluid and drug administration should be close to +10 mm filtration pressure or slightly above. Specialized pumps or other equipment can be used to deliver optimal pressure.

### Treatment of Dehydration and Volume Depletion Due to Trauma

Diarrheal disease is the second leading cause of death in children under five years old. Each year diarrhea kills around 525,000 children under the age of five in the developing world and the majority of these patients die from dehydration, according to the WHO.

The IV route is a very effective way for providing hydration or nutrition for patients who have severe dehydration; however, IV access is not always possible or feasible. For example, in cases when veins collapsed from dehydration, patients are agitated, or the patients have fragile veins. Qualified medical personnel is required to mountain venous access and prevent vein perforation and extravasation. In the case of an epidemic, in developing countries, there is often a shortage of qualified personnel to get and maintain IV access in a large number of patients simultaneously.

In these cases, alternative means of fluid delivery would be lifesaving. There are currently two potential alternatives routes for fluid administration: subcutaneous infusion and intraosseous infusion (administration of fluid inside the bone) R. Caccialanza et al, 2017. Intraosseous fluid administration requires highly-qualified personal and may result in bone fracture and osteomyelitis if done incorrectly. As a result, the subcutaneous infusion (or hypodermoclysis) has the benefit of being the most straightforward approach. Subcutaneous fluid administration is usually delivered by a gravity or infusion pump. Gravity infusion may help prevent local edema because the infusion rate naturally slows when the pressure in the subcutaneous space increases. The rate of infusion should remain within the limits of tissue perfusion. Usual infusion rate of 62 mL/h (or 1 cc/per minute) R. Caccialanza at al, 2017.

However, with severe dehydration, it is very important to administer a larger volume of fluids relatively quickly. As a result, fluid administration via an interstitial fluid reservoir would have the advantage over s/c fluid administration. The reservoir will allow large volume administration over a shorter period of time.

For severe dehydration initial bolus of fluid 20 ml/kg is recommended. The bolus of fluid is not possible with s/c fluid administration or interstitial reservoir. The only way to administer a bolus of fluid is iv. However, placing iv often takes time, particularly in severely dehydrated young patients as veins are collapsed because of dehydration. In comparison, fluid administration via reservoir can be started within 5 min and does not require nursing staff to maintain it. The collapse of the vein due to dehydration makes finding veins suitable for infusion difficult. Reservoir fluid administration will allow increased fluid administration for the first few hours of fluid administration to compensate for bolus. In the most severe cases, two reservoirs can be used.

If the patient weighs less than 10 kg, give 100 mL/kg/day. With iv administration patients that weigh 10 kg will receive 200 cc bolus and after bolus 42 cc/h (10 kg it will be 1000 ml/24 h 42 cc/h.)

The patient can receive 180 cc/h of fluid via reservoir (Table 1), so 200 cc bolus can be administered over 1.2 h followed by 42 cc/h of maintenance fluid.

If the patient weighs less than 20 kg, give 1000 mL/d plus 50 mL/kg/d for each kilogram between 10 and 20 kg. For example, for 20 kg kid 1,500 ml/24 h or 62.5 cc/h. The bolus 400 ml can be administered over a 2.5 h period.

If the patient weighs more than 20 kg, give 1500 mL/d, plus 20 mL/kg/d for each kilogram over 20 kg. For 30 kg the optimal fluid amount is 1700 ml/24 h or 70 cc/h. The bolus can be administered over a few hours, or a second reservoir can be used.

In the USA technology can be used in the case of mass casualties or military personnel injured during the war. In these cases, there would be a large number of individuals who need to have replaced the volume of lost blood, but there is not enough medical personnel to attend to all patients. In many cases, the correction of hypovolemia would be lifesaving. Shock severely decreases blood flow to subcutaneous tissue which negatively affects perfusion of subcutaneous tissues making s/c fluid administration much less feasible. As a result, the expandable reservoir would be a much better option and can be used immediately in the field.

### Total Parenteral Nutrition

Interstitial fluid administration can be beneficial for parenteral nutrition administration (TPN). In many chronically ill patients' veins are collapsing and these patients rely on central veins for both TPN and administration of other medications. Zaloga et al 2016 demonstrated that TPN can be administered s/c in elderly patients. However, only a limited volume of 62 ml/h can be administered s/c. Caccialanza et al 2016. However, the s/c line can easily dislodge with the movement of the patient so it could be only done in a setting where medical personnel is always available (nursing home or hospital vs patient's home). As a result, current guidelines recommend IV administration of TPN. The interstitial fluid via reservoir with interstitial fluid would be a good alternative for TPN administration. Using reservoir administration of TPN can also be used in the treatment of premature infants who are unable to take oral nutrition due to prematurity. Many of these babies are currently receiving TPN via central line (umbilical vein). However, umbilical vein catheterization has a high risk of complications. As with all central venous access, the complications of the placement include uncontrolled bleeding, infection, damage to adjacent structures, thrombosis, and placement into an artery. Specific to umbilical vein catheters, patients risk placement of the catheter into the portal venous system, which can lead to hepatic necrosis. Reports also describe liver abscess, portal vein thrombosis, and cavernoma formation. Due to the small volume of the neonate's venous system, central lines must be flushed with saline to assure no air is present in the line to prevent air embolism. Inadvertent placement in the umbilical artery can lead to catheter occlusion of limb arterial supply or thrombosis, leading to limb ischemia (Lewis et al 2020). Placement into the right atrium can lead to perforation and subsequent pericardial effusion. Overall, the complication rate of umbilical venous catheters is similar to that of percutaneously placed central venous catheters (Lewis et al 2020). In many cases using an interstitial fluid reservoir may be a better option.

### Administration of Medications

For patients, who need a central line because peripheral access is not available, using an interstitial fluid reservoir may be a better option in many cases. A central line (or central venous catheter) is like an intravenous (IV) line. It is much longer than a regular IV and goes all the way up to a vein near the heart or just inside the heart.) It also can be used to draw blood. The central line requires very qualified personnel (interventional radiologists) for placement. To prevent infection, tomb formation, and migration of the catheter into the location where it may cause a lot of harm central line has to be managed by very qualified personnel. However, in many cases for those who have no peripheral access, the central line is the only option that is currently available. In some of the cases, interstitial fluid reservoirs may substitute the need for central line placement. The interstitial fluid reservoir can also be used to substitute IV antibiotics to treat severe infections such as sepsis that require prolonged antibiotic use for 14 days or more for patients who don't have peripheral veins. Another indication woot an interstitial reservoir would be an administration of chemotherapy. Currently, chemotherapy in many cases has to be done in special infusion centers to ensure sterility and integrity of the central line in immunocompromised patients. The use of an interstitial reservoir instead of a central line would bring the cost of medical care down.

### Management of Pain Outside Hospital Settings

One of the most important pain management Options for pain is a Patient-Controlled Analgesia (PCA) that gives the patients ability to control the administration of medication and titrate administration of medication themselves depending on personal pain tolerance. That will be of the most important indication for the interstitial fluid reservoir will be the administration of medication for pain in patients in hospice patients. The patient-controlled analgesia (PCA) pump is a computerized machine that releases medication when a patient presses a button. PCA is self-administered on-demand analgesia. PCA is commonly used in the hospital to manage postoperative pain and pain caused by many diseases such as sickle cell or cancer. Autonomy through patient self-determination of the timing of analgesic administration is perhaps the fundamental advantage of PCA. Higher pain scores have previously been correlated with high levels of anxiety. Many patients experience an anxiety reduction and therefore have improved analgesia, because of the autonomy of PCA use. However, PCA needs to be connected to the IV and IV is very difficult to maintain at home which is why PCA is only available in the hospital or skilled nursing facility, but not at home.

In most cases, PCA pumps supply opioid pain-controlling drugs such as morphine, fentanyl, and hydromorphone. All these medications can be administered IM, IV, and SC so they can be also administered via an expandable reservoir.

Other medications could be administered via reservoir as well. For example, chemotherapy or medications to control seizures.

### Drug Implants

The intrathecal pump the implanted rechargeable drug delivery system that delivers medication to the spinal fluid is on the market for many years. However, no rechargeable pump delivers medication outside the intrathecal space. The scar tissue, connective tissue formation around the implant prevent precise administration of medication. Attachment of reservoir to the implantable technology would allow administration of medications in the interstitial fluid. This implantable rechargeable drug delivery system is important because it will be able to address compliance with medication.

The most important indication for the use of these types of implants is the treatment of substance use disorders (SUDs). In substance use disorders the disease itself will prevent patients from being compliant with treatment. For example, this technology will allow the development of a disulfiram implant, which will be a very effective and safe treatment for alcohol use disorder (AUD). However, the same technology can be used to administer other medications for the treatment of SUDs. In many cases, like disulfiram other forms of injectable depo formulations are not an option.

Many different embodiments have been disclosed herein, in connection with the above description and the drawings. It will be understood that it would be unduly repetitious and obfuscating literally to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support all claims to any such combination or subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A device utilized for the administration of medications, fluids, or parenteral nutrition, the device comprising:
a mesh insertable under the skin in a collapsed form, wherein the mesh is expanded and utilized with a stylet securely connected to an infusion system; and
a mechanism to secure a position within a reservoir utilized for administration of medications, a fluid, or parenteral nutrition.

2. The device of claim 1, wherein the mesh is expanded and utilized with a needle guided catheter.

3. The device of claim 1, wherein the reservoir is expanded via a balloon.

4. The device of claim 1, wherein the mesh is constructed of a bare metal or metal mesh.

5. The device of claim 1, wherein the mesh eludes a drug to decrease proliferation of interstitial tissue.

6. The device of claim 1, wherein the mesh of the reservoir is constructed from a biodegradable polymer.

7. The device of claim 6, wherein the mesh is covered by an enzyme hyaluronidase.

8. The device of claim 1, wherein the reservoir is used in conjunction with a patient-controlled anesthesia pump.

9. The device of claim 1, wherein the reservoir is used to treat dehydration

10. The device of claim 1, wherein the reservoir is used to treat hypovolemic shock.

11. The device of claim 1, wherein the stylet is secured by an inflatable balloon.

12. The device of claim 1, wherein the needle-guided catheter is secured by an inflatable balloon.

13. The device of claim 1, wherein the expendable reservoir is attached to a rechargeable implantable device to create an open space around the implantable device to administer one or more medications.

14. The device of claim 1, wherein the non-expendable reservoir is attached to a rechargeable implantable device to create an open space around the implantable device to administer one or more medications.

15. The device of claim 13, wherein the reservoir is used to administer one or more medications to treat substance use disorder.

16. The device of claim 1, wherein the rechargeable implantable device is used to administer naltrexone.

17. The device of claim 1, wherein the rechargeable implantable device is used to administer disulfiram.

18. The device of claim 1, wherein the reservoir and the catheter comprise a single device.
